(19) United States
(12) Patent Application Publication
(10) Pub. No.: US 2020/0338220 A1
(43) Pub. Date: Oct. 29, 2020

(54) UV-C Illumination System For Disinfection

(71) Applicant: Gerald Ho Kim, Ontario, CA (US)

(72) Inventor: Gerald Ho Kim, Ontario, CA (US)

(21) Appl. No.: 16/859,814

(22) Filed: Apr. 27, 2020

Related U.S. Application Data
(60) Provisional application No. 62/839,640, filed on Apr. 27, 2019.

(51) Int. Cl. A61L 2/10; B60S 1/64; B60H 1/00; A61L 2/24

(52) U.S. Cl. CPC A61L 2/10; B60S 1/64; A61L 2202/14; B60H 1/00814; A61L 2/24; B60H 1/00585

(57) ABSTRACT

An ultraviolet C (UV-C) illumination system includes a central disinfection unit (CDU), a heating, ventilation and air conditioning (HVAC) illuminator unit (HIU), and a beam scanning device. The CDU includes a fiber-coupled UV-C unit (FCU) configured to provide a UV-C light, a microcontroller, and a smart control unit (SCU) that controls each of the one or more FCUs by adjusting UV-C illumination by each of the one or more FCUs. The SMU communicates to the microcontroller to adjust energy consumption based on environmental data. The HIU includes fused silica rod to uniformly scatter the UV-C light to disinfect a surrounding area, with a light intensity controlled by the SCU of the CDU. The beam scanning device is coupled to the one or more FCUs. The beam scanning device includes a fiber and a lens disposed at a fiber tip of the fiber and configured to collimate the UV-C light.

### CROSS REFERENCE TO RELATED PATENT APPLICATION(S)

The present disclosure is a part of a non-provisional patent application claiming the priority benefit of U.S. Patent Application No. 62/839,640, filed on 27 Apr. 2019, the content of which being incorporated by reference in its entirety.

### TECHNICAL FIELD

The present disclosure generally relates to illumination laser and, more particularly, to ultraviolet C (UV-C) illumination for disinfection of interior and components of automobiles.

### BACKGROUND

Unless otherwise indicated herein, the approaches described in this section are not prior art to the claims in this application and are not admitted to be prior art by inclusion in this section.

One challenge of current approach for UV-C light-emitting diode (LED) illuminator used in automobile disinfection is cooling of the UV-C LED chip that is typically placed on the heating, ventilation and air conditioning (HVAC) system or the interior of automobile cabin. A low efficiency of current UV-C LED requires a good size of heatsink and air flow which limits feasibility in the design of a compact illuminator source.

### SUMMARY

The following summary is illustrative only and is not intended to be limiting in any way. That is, the following summary is provided to introduce concepts relating to a heat sink for thermal management in an electronic apparatus. Select embodiments of the novel and non-obvious technique are further described below in the detailed description. Thus, the following summary is not intended to identify essential features of the claimed subject matter, nor is it intended for use in determining the scope of the claimed subject matter.

In order to design a compact UV-C illuminator, a large heatsink and UV-C illuminator tip are separated by a fiber coupling method. The heatsink and UV-C LED chip of the UV-C illuminator are installed in a remote box with the UV-C light conveyed by a fiber made with fused silica-core. In this case the bulky heatsink and UV-C LED chip can be placed in a remote location and the fiber tip where the conveyed UV-C light gets out can be placed in the HVAC area and interior of an automobile. The fiber tip can be very small and compact to fit into any area or space and also does not require any heat dissipation. One main advantage of remotely separating UV-C LED and heatsink from the fiber tip is not only separating heat and UV-C light, but also the UV-C LED can be easily replaced if there is any issue or needs to be upgraded for higher UV-C power. If the UV-C LED is placed inside of HVAC and the roof of automobile cabin, it would be very difficult to replace the UV-C LED illuminator along the heatsink. The automobile disinfection design by separating the heatsink and UV-C LED source by fiber coupling design allows more flexible UV-C illuminator, easy to repair and easy to upgrade for higher power.

### BRIEF DESCRIPTION OF THE DRAWINGS

The accompanying drawings are included to provide a further understanding of the disclosure, and are incorporated in and constitute a part of the present disclosure. The drawings illustrate embodiments of the disclosure and, together with the description, serve to explain the principles of the disclosure. It is appreciable that the drawings are not necessarily in scale as some components may be shown to be out of proportion than the size in actual implementation in order to clearly illustrate the concept of the present disclosure.

FIG. 1 is a diagram of a concept of a central disinfection unit (CDU) in an automobile in accordance with an embodiment of the present disclosure.

FIG. 2 is a diagram of functional units related to a UV-C LED illuminator in accordance with an embodiment of the present disclosure.

FIG. 3 is a schematic diagram of a CDU in accordance with an embodiment of the present disclosure.

FIG. 4 is diagram of two fiber couple UV-C units (FCUs) in accordance with an embodiment of the present disclosure.

FIG. 5 is a diagram of a FCU connected to a HVAC illuminator in accordance with an embodiment of the present disclosure.

FIG. 6 is a schematic diagram of a CDU in accordance with an embodiment of the present disclosure.

FIG. 7 is a diagram of a HVAC illuminator in accordance with an embodiment of the present disclosure.

FIG. 8 is a diagram of a fused silica rod in accordance with an embodiment of the present disclosure.

### DETAILED DESCRIPTION OF PREFERRED EMBODIMENTS

### Overview

The present disclosure proposes various concepts, designs and embodiments of a device that provides a multi-illuminator design of UV-C LED to disinfect automobile interior and a HVAC heater-core area of the automobile. Current UV-C LED design tends to illuminate directly in the heater-core area of a HVAC system to disinfect all germs and mold, and the UV-C light can also illuminate the interior of automobile cabin. For instance, the UV-C LED illuminator can be placed in the HVAC area to shine the UV-C light directly to a heater-core where most of water condensation is accumulated during the operation of an air conditioner of the HVAC system. However, the condensed water or moisture in the HVAC tends to grow germs or mold quickly in the heater-core area due to humidity and warmer temperature once the air conditioner is off. To disinfect the HVAC area, the UV-C LED illuminator needs to be turned on for a certain duration to disinfect the germs and mold. Most of germs and molds are disinfected by UV-C light and odor from the germs and/or mold will be eliminated. Another application of automobile disinfection is decontamination of all germs and viruses in the interior of automobile cabin where human-touch or human airborne contamination can be spread. The UV-C light in the automobile cabin can be used to disinfect all the area effectively. One simple approach is to place UV-C LED to light up whole area of automobile cabin for disinfection. However, such approach requires many UV-C LEDs and costly wiring of all electrical connections.

The present disclosure aims to address the aforementioned issue by introducing a device that can effectively distribute UV-C light in both the HVAC system and interior of an automobile without increasing complexity of electrical wiring while allowing ease in installation and servicing components of UV-C illuminator. Under various proposed designs in accordance with the present disclosure, a Central Disinfection Unit (CDU) may have multiple units of UV-C LED illuminators with each illuminator connected to a respective fused silica-core to carry respective UV-C light to a respective fiber tip of the respective illuminator. One or more fiber tips may be routed to and disposed in the HVAC system for illuminating a heat-core area of the HVAC system. Likewise, one or more fiber tips may be routed to and disposed in the interior or cabin of the automobile for cabin disinfection. Under various proposed designs in accordance with the present disclosure, a combined fiber and heatsink module may form a Fiber Couple UV-C Unit (FCU), and multiple FCUs may be assembled to form a CDU. These FCUs may be controlled by a Smart Control Unit (SCU) in that the SCU may control each of the FCUs of the CDU to inject a determined or computed amount of UV-C illumination depending on sensor input (e.g., temperature and humidity) based on seasonal and geographical factors. It is important to feed correct information to the SCU so that the CDU can be operated with high efficiency. The SCU may receive information from a battery of the device as well as sensors that detect outside environmental factors. With the received information, the SCU may determine and control the UV-C LED power level and duration of illumination of the CDU. For instance, during the winter season, the CDU may be operated at a low power level and low duty cycle (or short duration of illumination) and, during the summer season, the CDU may be operated at a high power level and high duty cycle (or long duration of illumination). Also, power usage of the CDU may be based on the power level of an external battery. Under various proposed designs in accordance with the present disclosure, each fiber tip may be lensed and mounted on a canning system for better distribution of UV-C light. For instance, a beam scanning device may be mounted on or otherwise connected to the fiber tip to scan UV-C light. The UV-C light may also be lensed to deliver better-focused UV-C light in distance and may also be scanned.

One main issue on current UV-C illuminator in automobile is how to replace the illuminator module when it is faulty. The conventional way to replace a UV-C illuminator inside of HVAC system or to mount a UV-C illuminator inside an automobile cabin ceiling or dashboard is relatively risky since it tends to require a lot of disassembly time to remove the UV-C illuminator to be replaced. In contrast, by utilizing fiber optic with the CDU system in accordance with the present disclosure, the challenge or risk of extensive repair/replacement job for any faulty UV-C illuminator may be reduced. Another advantage of various designs in accordance with the present disclosure is the compactness of the UV-C illuminator. In the proposed designs, heatsink and UV-C LED may be placed in a CDU module so that there is no need for the fiber delivery system to carry any heat dissipating elements such as a fan or heatsink. Otherwise, in case that the UV-C illuminator is mounted directly inside the HVAC system or automobile cabin interior, the UV-C LED illuminator would need to be mounted on a heatsink to dissipate the heat generated by the UV-C LED where the UV-C LED generates about 95 to 98% of heat at present time. Thus, such a UV-C illuminator system design would become a huge burden with respect to mounting all heatsinks in HVAC system and automobile cabin interior. The CDU module according to a proposed design may have each UV-C illuminator mounted on a respective small heatsink and a single fan can cool all the UV-C illuminators. With such a centralized cooling scheme in the CDU, the proposed design allows easy power scaling of the UV-C illuminator as it can be easily replaced with or upgraded to a higher-power UV-C illuminator in the future.

Once UV-C LED light is fiber-coupled, its light can be transported to any place in the automobile such as its interior or its HVAC system. In order to disinfect any area inside the automobile the UV-C light needs to be distributed in the targeted area(s). The UV-C light may be combined with lenses to focus down to a desired locale such as, for example and without limitation, door handle, steering wheel and front dashboard. The fiber coupled light may be illuminated naturally or lensed to focus delivery or highly intense UV-C light. As the effectiveness of disinfection is directly proportional to the exposure dosage of UV-C light, the focused fiber beam in the proposed designs may disinfect much faster than if the UV-C light is natural dispersion out of the fiber.

Another method of delivering uniform dispersion of UV-C light may be to couple into a quartz, sapphire, or UV-C transmitting polymer rod. That is, the UV-C light coming out of the fiber end may be coupled into a rod. Some of the UV-C light may escape from the side of the rod and it may travel to the end of the rod. This is because the UV-C light coming out of the fiber has a high dispersion angle and the rod provides a wave-guide function to extend the UV-C light to travel at the end of the rod. In this case, a high intensity of UV-C light may be delivered to the end of the rod; otherwise, the UV-C light may be dispersed to a low-intensity beam at a short distance. This kind of dispersion method may be highly effective in HVAC system where the UV-C light needs to gradually spread around the entire area of the HVAC system.

In various proposed designs in accordance with the present disclosure, the amount of dispersion and spread of the UV-C light may be controlled by reflective or anti-reflective coating on the rod. This coating may allow controlling of the illumination intensity of the UV-C light surrounding of the rod. Moreover, UV-light emitted through the pattern or texture of the coating may generate illumination pattern to disinfect particular area faster than others due to high intensity.

Due to a remote placement of the UV-C illuminator by fiber coupling, a fiber coupled UV-C illuminator in the proposed designs may be used in other HVAC systems such as, for example and without limitation, in hospitals, homes, offices, warehouses, factories and anywhere applicable. For instance, the UV-C light coming out of or emitted from the fiber coupled rod may be placed in front of an air filter area of a hospital and home. In this case, the service and maintenance may be easy and may take place at the CDU as described above. Advantageously, replacing or maintaining the UV-C illuminator used in disinfecting a HVAC system in automobile, hospital, home and other applications may be performed with ease.

### Illustrative Implementations

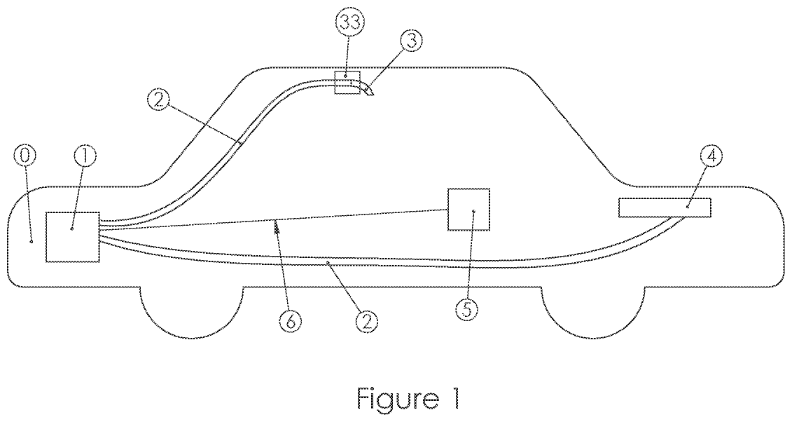

FIG. 1 illustrates a concept of a central disinfection unit (CDU) in an automobile in accordance with an embodiment of the present disclosure. Referring to FIG. 1, CDU 1 may be placed in a suitable location in an automobile (e.g., in a trunk or back-side of rear passenger seat). The fiber coupled UV-C LED may be carried by UV-C light transmitting fibers 2 and lens assembly 3 along with a scanning device 33 to the top of automobile cabin interior and a HVAC system (herein interchangeably referred to as HVAC Illuminator Unit (HIU) 4). The fibers 2 may passively deliver UV-C LED light to designated areas. The CDU 1 may be connected to an automobile Microcontroller Unit (MCU) 5 through an electrical wire 6 to communicate various status of automobile information. Due to high energy consumption of the CDU 1 and lifetime preservation, the CDU 1 may be controlled depending on temperature, humidity, dew-point and battery status. For example, the CDU 1 may be running at low energy consumption mode when the automobile is parked for long time and it may adjust its turn-on time depending on the battery charging level. If the automobile is operating during winter, the HVAC system or HIU 4 may run at very low operating duty cycle since no intense disinfection is needed in the HVAC system area.

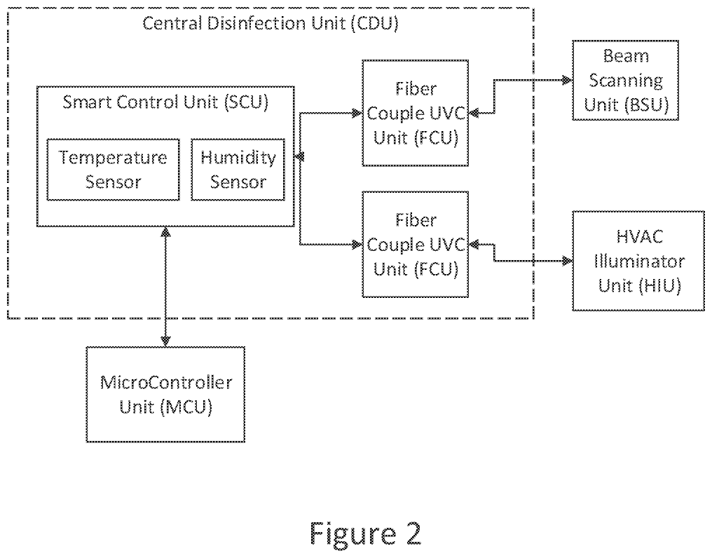

FIG. 2 illustrates functional units related to a UV-C LED illuminator in accordance with an embodiment of the present disclosure. FIG. 2 shows interconnection between all control units to UV-C LED illuminators. The CDU may contain a single Smart Control Unit (SCU) and multiple Fiber Couple UV-C Units (FCUs). One of the FCUs may be connected to a Beam Scanning Unit (BSU) and the another FCU may be connected to a HVAC Illuminator Unit (HIU). Also, the SCU may be connect to a Micro Controller Unit (MCU) for communication of automobile environmental data and battery charging level. The SCU may be equipped with sensors such as, for example and without limitation, a temperature sensor and a humidity sensor to control a fan to cool the FCUs. This control mechanism is needed to efficiently disinfect both HVAC and automobile cabin interior area. The system may operate in energy saving mode.

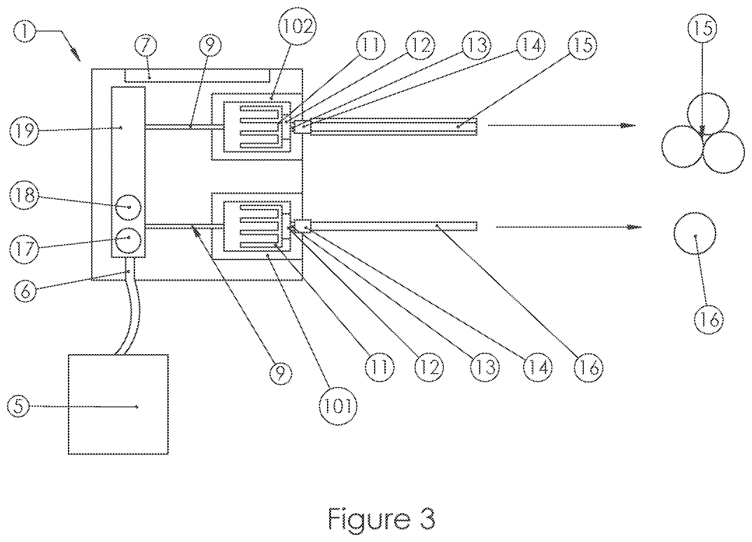

FIG. 3 illustrates a CDU 1 in accordance with an embodiment of the present disclosure. Referring to FIG. 3, CDU 1 may include two FCUs (e.g., FCU 101 and FCU 102). FCU 101 and FCU 102 may be connected to a SCU 19 along with a temperature sensor 17 and a humidity sensor 18 by electrical wiring 9. The SCU 19 may be also connected to an automobile MCU 5 along with an electrical wiring 6. FCU 101 may be configured with a single fiber carrying UV-C LED light to a disinfection area. FCU 102 may be configured with three fibers carrying UV-C LED light to a disinfection area. Each fiber may be connected to a respective UV-C LED light for high coupling efficiency. Due to the relatively large amount of heat generated by the CDU 1, a fan 7 may be installed in the system for cooling FCU 101 and FCU 102. FCU 101 may include a UV-C LED 13, a heat spreader 12, and a heatsink 11. FCU 102 may include three UV-C LEDs 13, three heat-spreaders 12 and one heatsink 11. FCU 101 may be connected with a UV-C light transmitting fiber 16 by a fiber connector 14. FCU 102 may be connected with three separate UV-C light transmitting fibers 15 by three fiber connectors 14. End views of the fiber 15 and fiber 16 are shown on the righthand side of FIG. 3.

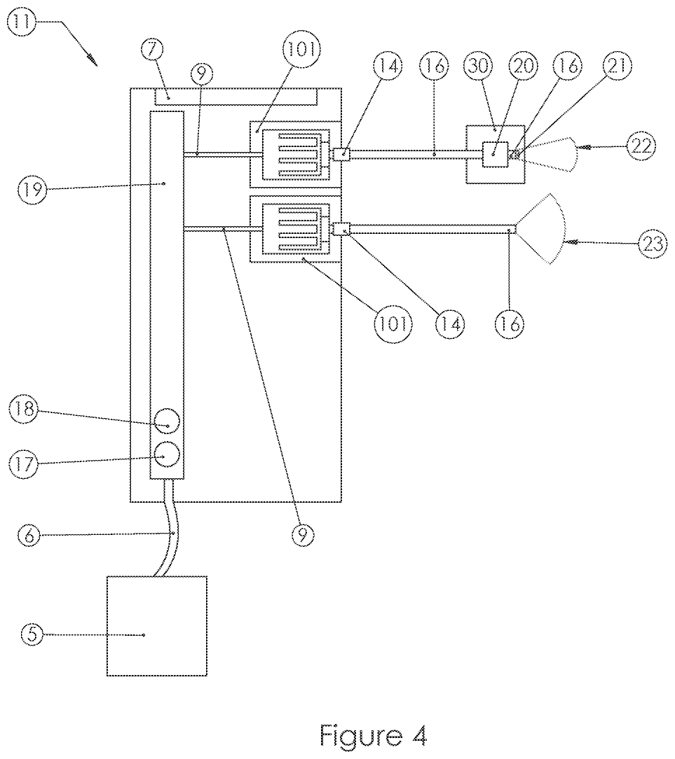

FIG. 4 illustrates two FCUs 101 in accordance with an embodiment of the present disclosure. Referring to FIG. 4, one of the FCUs 101 may have a large beam divergence angle 23 defined by a Numerical Aperture (NA) of a fiber 16. The fiber 16 of the other FCU 101 may be combined with a collimating lens 21 and a beam scanning device 20 to form a Bean Scanning Unit (BSU) 30. BSU 30 may be configured to tightly focus a fiber light 22, and the UV-C light may be conveyed by the beam scanning device 20. BSU 30 may effectively disinfect a small and long-range area in the automobile interior or any other surfaces.

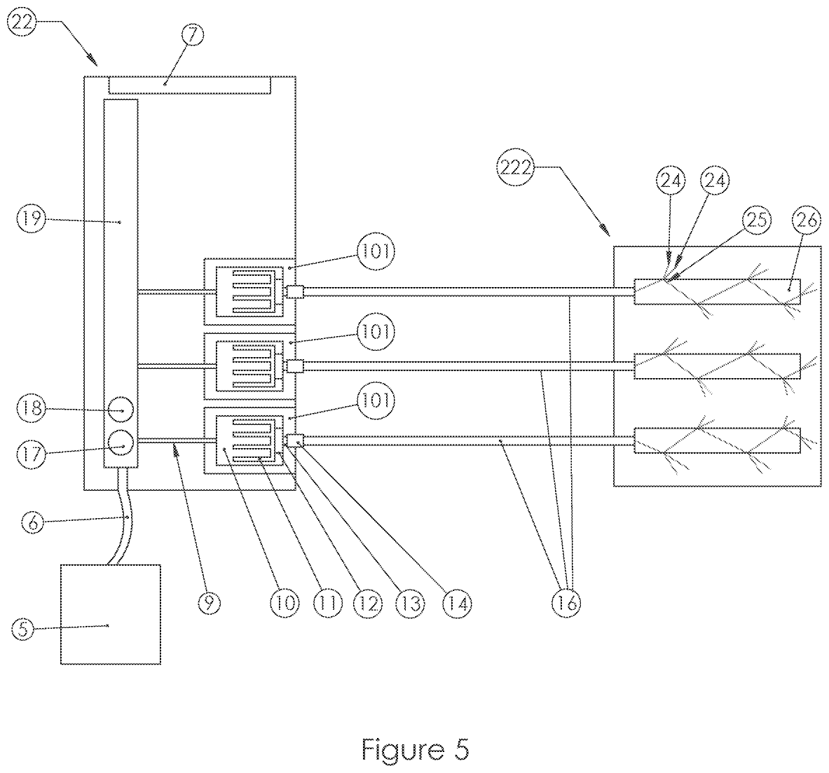

FIG. 5 illustrates a FCU 101 connected to a HVAC illuminator 222 in accordance with an embodiment of the present disclosure. Referring to FIG. 5, FCU 101 may be connected in HVAC illuminator 222. Fibers 16 may be connected to fused silica rods 26 by a butt-couple configuration and UV-C light may be scattered inside the fused silica rods 26. The UV-C light may be reflected and transmitted by the fused silica rods 2. In FIG. 5, the UV-C light is shown as a reflected beam 25 and a transmitted beam 24. Unlike fiber 16 that guides all UV-C light to the end of fiber 16, each fused silica rod 26 may leak the UV-C light with multiple bounces (shown as reflected beam 25 in FIG. 5). This phenomenon may create a uniform illumination around the fused silicon rods 26 for disinfecting the surrounding area in the HVAC system.

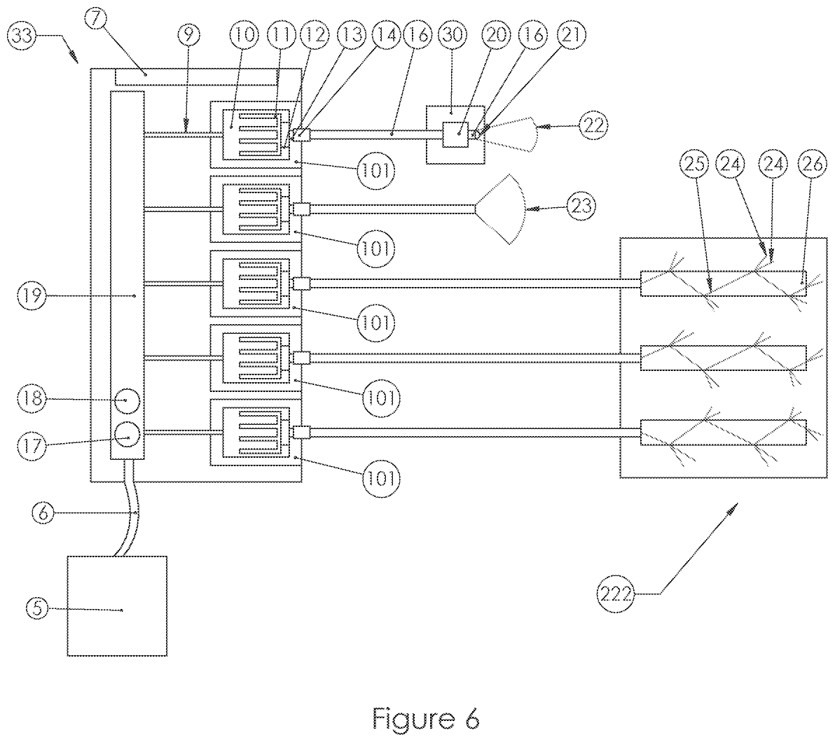

FIG. 6 illustrates a CDU 33 in accordance with another embodiment of the present disclosure. Referring to FIG. 6, a CDU 33 may include multiple (e.g., more than two) FCUs 101. All detailed descriptions above with respect to the examples shown in FIG. 4 and FIG. 5 are applicable to the example shown in FIG. 6. Thus, in the interest of brevity and to avoid redundancy, a detailed description of CDU 33 is not provided.

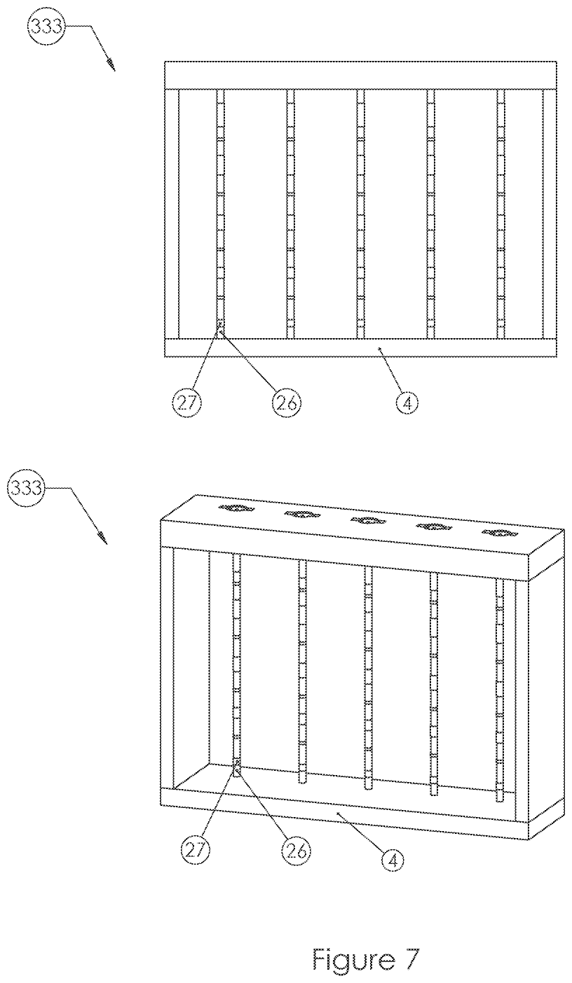

FIG. 7 is a diagram of a HVAC illuminator 333 in accordance with an embodiment of the present disclosure. Specifically, FIG. 7 shows a front view and a back side view of HVAC illuminator 333. In HVAC illuminator 333, each fused silica rod 26 may be contained in a frame 4 and the fused silica rod 26 may have scattering surface textures 27 designed for more light extraction through the texture 27. The FCU may be butt-coupled to the end of the fused silica rod 26 to uniformly illuminate the fused silica rod 26 for even distribution of UV-C illumination.

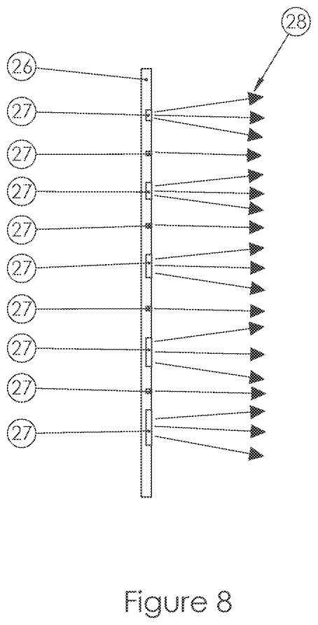

FIG. 8 is a diagram of a fused silica rod 26 in accordance with an embodiment of the present disclosure. Referring to FIG. 8, the fused silica rod 26 may be configured with various scatter surface textures 27 and UV-C light patterns 28. The textures 27 may be designed to bring more uniform illumination 28 from the fused silica rod 26 in particular direction where UV-C light is uniformly distributed. The fused silica rod 26 may also be replaced with other materials such as UV-C transmitting plastic or sapphire rod.

### Highlight of Select Features

In view of the above, select features in accordance with the present disclosure are highlighted below.

In one aspect, a UV-C illumination system may include a CDU, a HIU, and a beam scanning device. The CDU may include one or more FCUs configured to provide a UV-C light. The CDU may also include a microcontroller. The CDU may further include a smart control unit (SCU) that may be configured to control each of the one or more FCUs by adjusting UV-C illumination by each of the one or more FCUs. The SMU may also be configured to communicate to the microcontroller to adjust energy consumption based on environmental data. The HIU may include one or more fused silica rods to uniformly scatter the UV-C light to disinfect a surrounding area, with a light intensity of the UV-C light controlled by the SCU of the CDU. The beam scanning device may be coupled to the one or more FCUs. The beam scanning device may include a fiber moving device that includes a fiber and a lens disposed at a fiber tip of the fiber and configured to collimate the UV-C light.

In some implementations, the CDU may be connected to the HIU and the beam scanning device by one or more UV-C light transmitting fibers.

In some implementations, each of the one or more fibers may be made of fused silica.

In some implementations, the CDU may include a plurality of FCUs configured to disinfect a plurality of areas simultaneously.

In some implementations, an intensity of the UV-C light may be scalable when the one or more FCUs comprise a plurality of FCUs. In such cases, the UV-C light may be scaled up by coupling UV-C lights emitted by more than one FCUs of the plurality of FCUs together, with each of the plurality of FCUs independently controlled by the SCU.

In some implementations, the SCU may be connected to a temperature sensor and a humidity sensor to receive temperature data and humidity data, respectively, as the environmental data to operate in an energy-saving mode.

In some implementations, the SCU may be configured to control the one or more FCUs to adjust an intensity or duty cycle of the UV-C light based on battery power level information from the microcontroller and the environmental data.

In some implementations, the one or more FCUs may include a plurality of FCUs connected to a single fused silica rod of the one or more fused silica rods to increase UV-C light intensity.

In another aspect, a UV-C illumination system may include a CDU, a HIU, and a beam dispersion device. The CDU may include one or more FCUs configured to provide a UV-C light. The CDU may also include a microcontroller. The CDU may further include a smart control unit (SCU) that may be configured to control each of the one or more FCUs by adjusting UV-C illumination by each of the one or more FCUs. The SMU may also be configured to communicate to the microcontroller to adjust energy consumption based on environmental data. The HIU may include one or more fused silica rods to uniformly scatter the UV-C light to disinfect a surrounding area, with a light intensity of the UV-C light controlled by the SCU of the CDU. The beam dispersion device may be coupled to the one or more FCUs. The beam dispersion device may include a rod and a fiber connected to the rod and configured to leak the UV-C light from a side of the rod and to transport the UV-C light to an end of the rod. The rod may be patterned and textured to control intensity patterns of the UV-C light leaked to a surrounding of the rod.

In some implementations, the rod may include a patterned or textured quartz. Alternatively, the rod may include a patterned or textured sapphire. Alternatively, the rod may include a patterned or textured UV-C transmitting polymer.

In some implementations, the patterned or textured rod may be coated with a reflective film or an anti-reflective film.

In some implementations, the UV-C illumination system may be implemented inside an automobile to disinfect an interior and a HVAC system of the automobile.

In some implementations, the UV-C illumination system may be implemented inside a HVAC system of a medical facility (e.g., hospital).

In some implementations, the UV-C illumination system may be implemented inside an air duct or a HVAC system of a home, office, factory or warehouse.

### ADDITIONAL NOTES AND CONCLUSION

The herein-described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Further, with respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims, e.g., bodies of the appended claims, are generally intended as “open” terms, e.g., the term “including” should be interpreted as “including but not limited to,” the term “having” should be interpreted as “having at least,” the term “includes” should be interpreted as “includes but is not limited to,” etc. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an,” e.g., “a” and/or “an” should be interpreted to mean “at least one” or “one or more;” the same holds true for the use of definite articles used to introduce claim recitations. In addition, even if a specific number of an introduced claim recitation is explicitly recited, those skilled in the art will recognize that such recitation should be interpreted to mean at least the recited number, e.g., the bare recitation of “two recitations,” without other modifiers, means at least two recitations, or two or more recitations. Furthermore, in those instances where a convention analogous to “at least one of A, B, and C, etc.” is used, in general such a construction is intended in the sense one having skill in the art would understand the convention, e.g., “a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, e.g., “a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. An ultraviolet C (UV-C) illumination system, comprising:
a central disinfection unit (CDU), the CDU comprising:
one or more fiber-coupled UV-C units (FCUs) configured to provide a UV-C light;
a microcontroller; and
a smart control unit (SCU) configured to control each of the one or more FCUs by adjusting UV-C illumination by each of the one or more FCUs, the SMU further configured to communicate to the microcontroller to adjust energy consumption based on environmental data;
a heating, ventilation and air conditioning (HVAC) illuminator unit (HIU) comprising one or more fused silica rods to uniformly scatter the UV-C light to disinfect a surrounding area, with a light intensity of the UV-C light controlled by the SCU of the CDU; and
a beam scanning device coupled to the one or more FCUs, the beam scanning device comprising a fiber moving device that comprises:
a fiber; and
a lens disposed at a fiber tip of the fiber and configured to collimate the UV-C light.

2. The UV-C illumination system of claim 1, wherein the CDU is connected to the HIU and the beam scanning device by one or more UV-C light transmitting fibers.

3. The UV-C illumination system of claim 1, wherein each of the one or more fibers is made of fused silica.

4. The UV-C illumination system of claim 1, wherein the CDU comprises a plurality of FCUs configured to disinfect a plurality of areas simultaneously.

5. The UV-C illumination system of claim 1, wherein an intensity of the UV-C light is scalable when the one or more FCUs comprise a plurality of FCUs, wherein the UV-C light is scaled up by coupling UV-C lights emitted by more than one FCUs of the plurality of FCUs together, and wherein each of the plurality of FCUs is independently controlled by the SCU.

6. The UV-C illumination system of claim 1, wherein the SCU is connected to a temperature sensor and a humidity sensor to receive temperature data and humidity data, respectively, as the environmental data to operate in an energy-saving mode.

7. The UV-C illumination system of claim 6, wherein the SCU is configured to control the one or more FCUs to adjust an intensity or duty cycle of the UV-C light based on battery power level information from the microcontroller and the environmental data.

8. The UV-C illumination system of claim 1, wherein the one or more FCUs comprise a plurality of FCUs connected to a single fused silica rod of the one or more fused silica rods to increase UV-C light intensity.

9. An ultraviolet C (UV-C) illumination system, comprising:
a central disinfection unit (CDU), the CDU comprising:
one or more fiber-coupled UV-C units (FCUs) configured to provide a UV-C light;
a microcontroller; and
a smart control unit (SCU) configured to control each of the one or more FCUs by adjusting UV-C illumination by each of the one or more FCUs, the SMU further configured to communicate to the microcontroller to adjust energy consumption based on environmental data;
a heating, ventilation and air conditioning (HVAC) illuminator unit (HIU) comprising one or more fused silica rods to uniformly scatter the UV-C light to disinfect a surrounding area, with a light intensity of the UV-C light controlled by the SCU of the CDU; and
a beam dispersion device coupled to the one or more FCUs, the beam dispersion device comprising:
a rod; and
a fiber connected to the rod and configured to leak the UV-C light from a side of the rod and to transport the UV-C light to an end of the rod,
wherein the rod is patterned and textured to control intensity patterns of the UV-C light leaked to a surrounding of the rod.

10. The UV-C illumination system of claim 9, wherein the rod comprises a patterned or textured quartz.

11. The UV-C illumination system of claim 9, wherein the rod comprises a patterned or textured sapphire.

12. The UV-C illumination system of claim 9, wherein the rod comprises a patterned or textured UV-C transmitting polymer.

13. The UV-C illumination system of claim 9, wherein the patterned or textured rod is coated with a reflective film or an anti-reflective film.
